(19) United States
(12) Patent Application Publication
(10) Pub. No.: US 2016/0113545 A1
(43) Pub. Date: Apr. 28, 2016

(54) SYSTEM AND METHOD FOR ANALYZING ELECTROENCEPHALOGRAM IN RESPONSE TO IMAGE STIMULUS OF MEDIA FACADE

(71) Applicant: Foundation of Soongsil University-Industry Cooperation, Seoul (KR)

(72) Inventors: Juyeon KIM, Seoul (KR); Jongha KIM, Yeongju-si (KR); Sanghee KIM, Jecheon-si (KR); Jeongho LEE, Daegu (KR)

(21) Appl. No.: 14/854,834

(22) Filed: Sep. 15, 2015

(30) Foreign Application Priority Data
Oct. 23, 2014 (KR) 10-2014-0144023

(51) Int. Cl. A61B 5/0484; A61B 5/00; A61B 5/04

(52) U.S. Cl. CPC A61B 5/04842; A61B 5/04012; A61B 5/742

(57) ABSTRACT

Provided are a system and method for analyzing an electroencephalogram (EEG) response to an image stimulus of a media facade which examine, with quantitative data, how emotional reactions of people are affected by image stimuli of different media facades. The system measures EEGs of subjects with respect to stimuli of different media facades and analyzes reaction characteristics, such as activities of respective EEG bands, activities of EEG bands according to brain regions, regional correlations according to the EEG bands, and differences in activities of EEG bands between male and female groups of subjects.

### STATEMENT REGARDING PRIOR DISCLOSURES BY THE INVENTORS

A journal article entitled “A Study on EEG Response to a Video Stimulus to Media Facades—Two Cases of the Galleria Department Store and the Seoul Square”, published on Sep. 30, 2014, does not qualify as prior art under AIA 35 U.S.C. 102(b)(1)(A) as the disclosure was made by the joint inventors. A copy of the thesis paper is submitted herewith in an Information Disclosure Statement.

### CROSS-REFERENCE TO RELATED APPLICATION

This application claims priority to and the benefit of Korean Patent Application No. 10-2014-0144023, filed on Oct. 23, 2014, the disclosure of which is incorporated herein by reference in its entirety.

### BACKGROUND

The present disclosure relates to a system and method for analyzing an electroencephalogram (EEG) response, and more particularly, to a system and method for analyzing an EEG response to an image stimulus of a media facade which examine, with quantitative data, how emotional reactions of people are affected by image stimuli of different media facades.

The development of information technology (IT) has been grafted onto the diversity of building skins, and modern architecture is being developed not only to provide the feeling of visual satisfaction but also to both influence and be affected by urban space, stimulate emotions of people, and interact with people.

While the value of emotions and the importance of emotional design are under active discussion these days, architectural design is also being developed to follow the trends.

A typical example of such architectural design is a media facade.

Media facades are examples of actively using visual media and content by providing screens on building skins.

The term “Media facade” is a combination of media and a facade which means an outer wall of a building, and implies projection of a three-dimensional (3D) image on an inner or outer wall of a building used as a screen.

In particular, media facades are highly recognizable and attract wide attention at a long distance and thus are easily exposed to the public.

In connection with media facades, it is necessary to discuss the positive effects of providing a little experience of an art work in daily life and stimulating emotions in a desolate urban space and negative effects of giving visual displeasure or causing stress.

This is because a visual stimulus coming from the surroundings triggers a variety of psychological mechanisms of people.

Thus far, many scholars have researched the influence of image stimuli on the psychology and behavior of people, and have verified the fact that there is a clear relationship between an emotional reaction to a stimulus and cerebral activation.

Therefore, it is important to know how a large number of urban people who are unselectively exposed to various content of media facades in urban space are affected psychologically and physiologically.

Referring to previous research which has been conducted thus far in Korea with regard to media facades, there have been attempts to measure emotions based on the assumption that a media facade may be a medium for influencing and being affected by people, but most attempts have been limited to emotional evaluation or satisfaction research based on surveys.

Such a research method has a disadvantage in that it is difficult to control extraneous variables, such as a memory corruption or distortion, through an unconscious process.

### SUMMARY OF THE DISCLOSURE

According to one embodiment of the present disclosure, a system for analyzing an electroencephalogram (EEG) reaction to an image stimulus of a media facade is provided. The system includes an EEG sensing unit configured to measure EEG signal strengths emitted from a plurality of EEG electrode channels installed in a plurality of regions of a brain of a viewer who views an experimental image stimuli, a fast Fourier transform (FFT) unit configured to convert the EEG signal strengths from a time domain to a frequency domain, a control unit configured to extract wavelengths in a predetermined frequency band from the frequency domain of the EEG signal strengths and to extract a power amount for each frequency using a frequency-specific power spectrum, and an EEG band activity analysis unit configured to calculate a first EEG average in response to EEG activity ratios per an EEG band with respect to an average of a band-to-band power value based on the power amount for each frequency, to calculate a second EEG average of the EEG electrode channels in response to the EEG band according to the plurality of regions of the brain, and to comparatively analyze brain-region-specific activity ratios of the EEG band.

The EEG band includes a plurality of EEG frequencies including a relative theta wave of 4 to 8 Hz, a relative alpha wave of 8 to 13 Hz, a relative beta wave of 13 to 30 Hz, and a relative gamma wave equal or greater than 30 Hz in ascending order of frequency, and the EEG electrode channels includes a left prefrontal lobe, a right prefrontal lobe, a left frontal lobe, a right frontal lobe, a left parietal lobe, a right parietal lobe, a left occipital lobe, and a right occipital lobe.

The experimental image stimuli are three-dimensional (3D) images projected onto an inner or outer wall of a building, and include a first image stimulus configured with a pattern change in color and shape and a second image stimulus configured to use a change in color and shape for delivering a story as a storytelling medium.

The EEG band activity analysis unit is configured to determine brain-region-specific activities by calculating the second EEG average of the EEG electrode channels depending on the EEG band according to the first image stimulus or the second image stimulus, and to analyze image stimulus results of the first image stimulus or the second image stimulus exerted on the viewer by analyzing changes in and differences between the second EEG average of the EEG electrode channels resulting from the first image stimulus or the second image stimulus.

The EEG band activity analysis unit is configured to analyze correlations between the second EEG average of the EEG electrode channels and to determine that the prefrontal lobes are brain regions having a correlation coefficient of 0.9 or more between EEG electrode channels with respect to the first image stimulus and the second image stimulus.

The EEG band activity analysis unit is configured to analyze the second EEG average of the EEG electrode channels and determine that a correlation between left and right cerebral hemispheres is shown in a relative alpha wave band rather than relative theta, beta, and gamma wave bands among brain regions showing a correlation coefficient of 0.7 or more between the EEG electrode channels with respect to the first image stimulus and the second image stimulus.

At least one of the first EEG average and the second EEG average is calculated by subtracting image stimulus EEG averages of the experimental image stimuli from a background EEG average indicating a base EEG average resulting from a white image stimulus.

According to another embodiment of the present disclosure, a method of analyzing an electroencephalogram (EEG) reaction to an image stimulus of a media facade is provided. The method includes measuring EEG signal strengths from a plurality of EEG electrode channels installed in a plurality of regions of a brain of a viewer who views an experimental image stimuli, converting the EEG signal strengths from a time domain to a frequency domain using a fast Fourier transform (FFT) method, extracting wavelengths in a predetermined frequency band from the frequency-domain of the EEG signal strengths, and extracting a power amount for each frequency using a frequency-specific power spectrums, calculating a first EEG average in response to EEG activity ratios per an EEG band with respect to an average of a band-to-band power value based on the power amount for each frequency, calculating a second EEG average of the EEG electrode channels in response to the EEG band according to the plurality of regions of the brain, and comparatively analyzing brain-region-specific activity ratios of the EEG band.

The EEG band includes a plurality of EEG frequencies including a relative theta wave of 4 to 8 Hz, a relative alpha wave of 8 to 13 Hz, a relative beta wave of 13 to 30 Hz, and a relative gamma wave equal or greater than 30 Hz in ascending order of frequency, and the EEG electrode channels includes a left prefrontal lobe, a right prefrontal lobe, a left frontal lobe, a right frontal lobe, a left parietal lobe, a right parietal lobe, a left occipital lobe, and a right occipital lobe.

The experimental image stimuli are three-dimensional (3D) images projected onto an inner or outer wall of a building, and include a first image stimulus configured with a pattern change in color and shape and a second image stimulus configured to use a change in color and shape for delivering a story as a storytelling medium, brain-region-specific activities are determined by calculating the second EEG average of the EEG electrode channels depending on the EEG band according to the first image stimulus or the second image stimulus, and image stimulus results of the first image stimulus or the second image stimulus exerted on the viewer by analyzing changes in and differences between the second EEG average of the EEG electrode channels resulting from the first image stimulus or the second image stimulus.

The calculating of the first EEG average includes generating the first image stimulus and the second image stimulus resulted from a highest EEG average of the left frontal lobe among the brain regions in a relative theta wave band, and resulted from a lowest EEG average of the left frontal lobe and a highest EEG average of the right occipital lobe among the brain regions in a relative gamma wave band, and brain mapping image data visually showing which region of the brain has been activated is generated.

The comparative analyzing of the brain-region-specific activity ratios includes analyzing correlations between the second EEG average of the EEG electrode channels, and finding that the prefrontal lobes are brain regions having a correlation coefficient of 0.9 or more between EEG electrode channels with respect to the first image stimulus and the second image stimulus.

### BRIEF DESCRIPTION OF THE DRAWINGS

The patent or application file contains at least one drawing/photograph executed in color. Copies of this patent or patent application with color drawing(s)/photograph(s) will be provided by the Office upon request and payment of the necessary fee.

The above and other objects, features and advantages of the present disclosure will become more apparent to those of ordinary skill in the art by describing in detail exemplary embodiments thereof with reference to the accompanying drawings, in which:

FIG. 1 is a table showing examples of media facades according to an exemplary embodiment of the present disclosure;

FIG. 2 is a schematic diagram of a system for analyzing an electroencephalogram (EEG) response to an image stimulus of a media facade according to an exemplary embodiment of the present disclosure;

FIG. 3 is a block diagram showing a schematic configuration of an EEG response analysis unit in a system for analyzing an EEG response according to an exemplary embodiment of the present disclosure;

FIG. 4 is a diagram showing brain mapping image data of EEG bands dependent on image stimuli according to an exemplary embodiment of the present disclosure;

FIG. 5 is a histogram showing changes in activities of EEG bands dependent on brain regions according to an exemplary embodiment of the present disclosure;

FIG. 6 is a diagram in which correlations between brain regions with respect to an image stimulus of a Galleria department store are analyzed according to an exemplary embodiment of the present disclosure;

FIG. 7 is a diagram in which correlations between brain regions with respect to an image stimulus of Seoul Square are analyzed according to an exemplary embodiment of the present disclosure;

FIG. 8 is a diagram showing differences in EEG activity caused by the Galleria Dept. image stimulus between male and female groups according to an exemplary embodiment of the present disclosure;

FIG. 9 is a table of EEG-band-specific brain mapping image data resulting from the Galleria Dept. image stimulus according to an exemplary embodiment of the present disclosure;

FIG. 10 is a diagram showing differences in EEG activities caused by the Seoul Square image stimulus between male and female groups according to an exemplary embodiment of the present disclosure;

FIG. 11 is a table of EEG-band-specific brain mapping image data resulting from the Seoul Square image stimulus according to an exemplary embodiment of the present disclosure;

FIG. 12 is a flowchart illustrating a method of analyzing an EEG response to an image stimulus of a media facade according to an exemplary embodiment of the present disclosure; and

FIG. 13 is a table showing comprehensively arranged EEG analysis results of subjects with respect to the Galleria Dept. and Seoul Square image stimuli according to an exemplary embodiment of the present disclosure.

### DETAILED DESCRIPTION OF EXEMPLARY EMBODIMENTS

Advantages and features of the present disclosure and a method of achieving the same will be more clearly understood from embodiments described below in detail with reference to the accompanying drawings. However, the present disclosure is not limited to the following embodiments and may be implemented in various different forms. The embodiments are provided merely for complete disclosure of the present disclosure and to fully convey the scope of the disclosure to those of ordinary skill in the art to which the present disclosure pertains. The present disclosure is defined only by the scope of the claims. Throughout this specification, like reference numerals refer to like elements.

The terminology used herein is for describing particular embodiments only and is not intended to be limiting of the present disclosure. As used herein, the singular forms “a,” “an,” and “the” are intended to include the plural forms as well, unless the context clearly indicates otherwise. It will be further understood that the terms “comprises,” “comprising,” “includes,” and/or “including,” when used herein, specify the presence of stated components, steps, operations, and/or elements, but do not preclude the presence or addition of one or more other components, steps, operations, and/or elements.

Embodiments of the present disclosure measure brain reactions to image stimuli of examples of Korean media facades using a method of measuring an electroencephalogram (EEG) of a subject with respect to an image stimulus among a variety of physiological measurement methods, and analyze EEG reaction characteristics and emotional changes through the derived quantitative data.

Embodiments of the present disclosure measure EEGs of a subject with respect to the image stimuli of examples of Korean media facades and analyze characteristics of response outcomes.

For the examples of the image stimuli, the Galleria department store and the Seoul Square, which have different content expression methods, are selected.

A person's perception of an external factor is transmitted to the brain through nerves, undergoes a complex and rapid process, and is housed in the limbic system which is in charge of emotions. Then, the limbic system generates a variety of hormones and various neurotransmitters through interactions between various neurons and effects of innumerable synapses.

Naturally, there is a change in the person's emotion, and in addition, a difference in strength of the sensory perception is generated. Through such physical reaction, emotions and behaviors of the person are changed.

With the disclosure of such principle, many attempts to physiologically measure the level of an emotion or psychological pleasure are being made.

To measure the emotions of a person, research is being conducted for analyzing the subjective psychology of the person as accurate and objective numerical values through measured values of event-related potentials (ERPs), an EEG, an electrocardiogram (ECG), etc.

In particular, an EEG monitoring device is reported to be the most effective among the various physiological measurement methods.

A brainwave is a generic term referring to a movement of a signal transferred between cerebral nerves.

To understand and analyze a brainwave, it is necessary to understand the functions of a cerebrum according to positions in the cerebrum and frequency-specific characteristics of the cerebrum. First, according to the positions and the functions, the cerebrum is divided into frontal lobes, temporal lobes, parietal lobes, and occipital lobes.

The frontal lobes are located at the front portion of the cerebrum, are in charge of functions of thinking, cognition, problem solving, etc., and are involved in attention concentration and behavioral control. The temporal lobes are located behind both ears, and are mainly involved in auditory perception and a linguistic capability.

The parietal lobes' function is to process physical information received from the sense of touch, stretch receptors, joint receptors, and so on.

The occipital lobes, in which a neuraxis of thalamic nuclei receiving an input from a visual pathway is present, are in charge of working memory for storing spatial information.

Meanwhile, brainwaves are classified in order of increasing frequency into delta waves, theta waves, alpha waves, beta waves, and gamma waves, and characteristics of the respective frequency bands are shown in Table 1 below.

TABLE 1

| | Frequency | | |
|---|---|---|---|
| Classification | band | | Related brain activity |
| Delta wave (δ) | 0.5 to 4 | Hz | Deep sleep |
| Theta wave (θ) | 4 to 8 | Hz | General sleep, drowsing, and |
| | | | meditation |
| Alpha wave (α) | 8 to 13 | Hz | Emotional stability, |
| | | | relaxation, and |
| | | | comfortable state |
| Beta wave (β) | 13 to 30 | Hz | Mental activity, |
| | | | consciousness, |
| | | | tense, and stress |
| Gamma wave (γ) | 30 | Hz or above | High-level information |
| | | | processing process, and |
| | | | complex mental activity |

Similar to the image of a media facade, receiving a visual stimulus from the surrounding means more than just looking at the visual stimulus. Visual information reaching the occipital lobes through the eyes, which are sensory organs, are edited through the adjustment of frontal-lobe or parietal-lobe circuits or the limbic system which is a part of the brain for emotional memory.

Also, in the process of processing a stimulus input from the outside, a variety of psychological mechanisms of people are triggered by several complex factors, such as the intervention of short-term memory, an emotional system, a management system, or long-term memory.

Expression types of media facades are classified according to a variety of references (materials or material quality, interaction methods, display technologies, etc.) for classification proposed by respective researchers.

In an early stage, content used in media facades has generally used graphic elements including symbols, illustrations, images, or color patterns and showing simplified movements, but is gradually extending its boundary to public art as content, such as art works of media artists, which is grafted onto the content used in media facades.

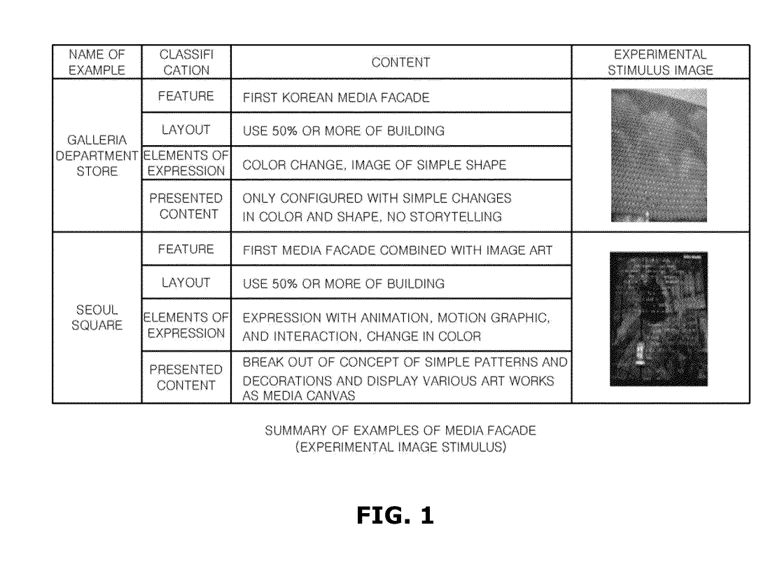

As shown in FIG. 1, an example of the former case is a Galleria department store, which is the first media facade to have been introduced in Korea. The media facade of the Galleria department store is a typical example that displays a simple change in color and an image of a shape. An example of the latter case is Seoul Square, which breaks out of the concept of simple patterns or decorations and aims for a media exhibition space for urban people by showing media images of famous Korean and foreign artists including the media art works of Julian Opie and Mankee Yang, and presenting the respective art works with various types of storytelling.

How different emotions of a subject result from the two examples which have different elements of expression and presented content as mentioned above will be analyzed based on a change in EEG.

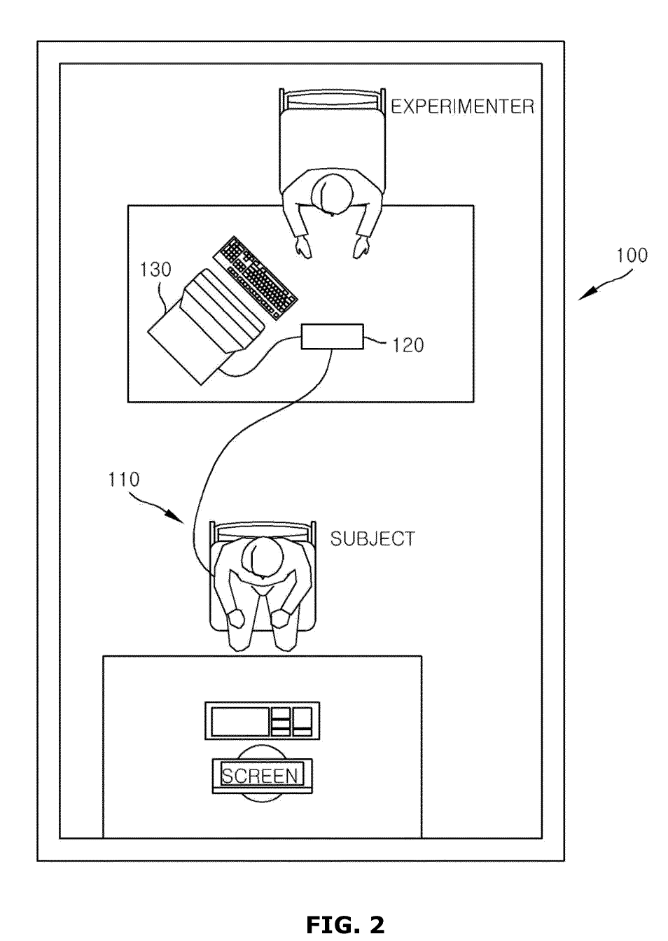

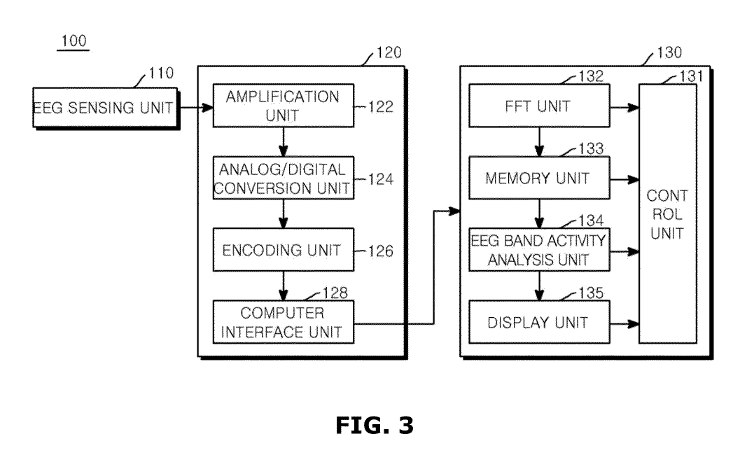

FIG. 1 is a table showing examples of media facades according to an exemplary embodiment of the present disclosure, FIG. 2 is a schematic diagram of a system for analyzing an EEG response to an image stimulus of a media facade according to an exemplary embodiment of the present disclosure, and FIG. 3 is a block diagram showing a schematic configuration of an EEG response analysis unit in a system for analyzing an EEG response according to an exemplary embodiment of the present disclosure.

A system 100 for analyzing an EEG response to an image stimulus of a media facade according to an exemplary embodiment of the present disclosure includes an EEG sensing unit 110, an interface unit 120, and an EEG response analysis unit 130.

The EEG sensing unit 110 senses EEG signals from 32 channel EEG electrodes attached to predetermined positions on a subject's scalp. Here, the 32 channel EEG electrodes are devices for selectively or simultaneously measuring EEGs at a measuring region among the prefrontal lobes, the frontal lobes, the parietal lobes, the temporal lobes, and the occipital lobes.

The interface unit 120 is electrically connected to the EEG sensing unit 110 through a wire and includes an amplification unit 122, an analog/digital conversion unit 124, an encoding unit 126, and a computer interface unit 128.

The amplification unit 122 filters weak EEG signals sensed by the EEG sensing unit 110 and then amplifies the amplitudes.

The analog/digital conversion unit 124 samples each of the amplified EEG signals of the plurality of channels hundreds of times per second and converts the sampled signals into digital values.

The encoding unit 126 sequentially encodes the identifiers of the respective channels and the digital values of channel-specific one bytes in real time.

The computer interface unit 128 transmits digital signals encoded by the encoding unit 126 to the EEG reaction analysis unit 130.

The EEG reaction analysis unit 130 includes a control unit 131, a fast Fourier transform (FFT) unit 132, a memory unit 133, an EEG band activity analysis unit 134, and a display unit 135.

The control unit 131 transmits the time-domain EEG signals received from the interface unit 120 to the FFT unit 132. Here, the time-domain EEG signals indicate EEG data of a left prefrontal lobe Fp1, a right prefrontal lobe Fp2, a left frontal lobe F3, a right frontal lobe F4, a left parietal lobe P3, a right parietal lobe P4, a left occipital lobe O1, and a right occipital lobe O2 among data of the 32 channels.

The FFT unit 132 converts the time-domain EEG signals received from the control unit 131 into frequency-domain signals.

The control unit 131 extracts wavelengths of a specific frequency band from the frequency-domain EEG signals converted by the FFT unit 132, extracts frequency-specific power amounts using frequency-specific power spectrums, and stores the extracted power amounts of the specific frequency band in the memory unit 133. Here, the wavelengths of the specific frequency band denote delta waves, theta waves, alpha waves, beta waves, and gamma waves as EEG frequencies in order of increasing frequency.

The EEG band activity analysis unit 134 calculates EEG-band-specific EEG averages, which represent EEG-band-specific activity ratios based on the average of band-to-band powers, based on the power amounts of the specific frequency band received from the control unit 131 (see Table 2 below).

The EEG band activity analysis unit 134 calculates EEG averages of the EEG electrode channels in the respective EEG bands according to the brain regions, and comparatively analyzes activity ratios of the EEG bands according to the brain regions.

The EEG band activity analysis unit 134 calculates the average and the standard deviation of subject-specific average band-to-band powers using SPSS 17.0, which is a statistical analysis program, and performs a correlation analysis, an independent sample T-test, and a paired sample T-test to comparatively analyze EEG change characteristics according to the image stimuli of media facades and differences in EEG activity between male and female groups.

As shown in FIG. 1, the experimental image stimuli are the media facades of the Galleria department store and Seoul Square.

In other words, an experimental image stimulus is a three-dimensional (3D) image projected on an inner or outer wall of a building used as a screen, and includes a first image stimulus (the Galleria department store) which is configured with a simple pattern of changes in color and shape and a second image stimulus (Seoul Square) which uses various changes in color and shape as a storytelling medium delivering a story.

An EEG average is obtained by subtracting an EEG average resulting from an image stimulus of a media facade from a background EEG average (a base EEG average resulting from a white image stimulus).

Data of a total of 56 subjects including 28 males and 28 females was analyzed. As shown in FIG. 2, after the 32 channel EEG electrodes were attached to the scalps of the subjects, the subjects sat on chairs to relax their breathing for five minutes while correcting their postures, and then the tests were started.

First, without any external stimulus, background (fundamental) EEGs, which are voluntary electrical activities of cerebral cortical neurons, were measured for one minute and 30 seconds and recorded, and then stopped for two minutes.

Next, the subjects viewed an image stimulus of the media facade of the Galleria department store for 10 seconds, and then their EEGs were measured. After a break, the subjects viewed an image stimulus of the media facade of Seoul Square for 10 seconds, and then their EEGs were measured.

Measuring of EEGs was started when stable EEGs without artifacts continued for 10 seconds or more.

The EEG band activity analysis unit 134 compares EEG-band-specific activity ratios according to experimental image stimuli, and analyzes EEG-band-specific changes based on the average of band-to-band powers to examine the subjects' brain reactions to the experimental image stimuli of the media facades.

TABLE 2
Comparison of EEG band activities between image stimuli (N = 56)

| | Stimulus-specific EEG averages (SD) | | | | |
|---|---|---|---|---|---|
| | (Background stimulus − image stimulus) | | | | |
| | Galleria department | | Difference in | | |
| Waveform | store | Seoul Square | average (D) | t value | p value |
| Relative theta | 0.023 (0.123) | 0.027 (0.123) | −0.004 | −0.218 | 0.828 |
| Relative alpha | 0.005 (0.131) | −0.022 (0.120) | 0.027 | 1.429 | 0.159 |
| Relative beta | −0.010 (0.065) | 0.000 (0.066) | −0.01 | −1.136 | 0.261 |
| Relative gamma | −0.018 (0.090) | −0.005 (0.077) | −0.013 | −1.035 | 0.305 |

As shown in Table 2 above, each EEG band had a difference in average, which is not statistically significant.

Referring to band-specific changes, a band showing the most activated EEG change corresponded to relative theta waves. The relative theta wave band was more activated by an image stimulus of the media facade of Seoul Square (referred to as “Seoul Square image stimulus” below) than by an image stimulus of the media facade of the Galleria department store (referred to as “Galleria Dept. image stimulus” below).

In the case of relative alpha waves, an EEG average was increased by the Galleria Dept. image stimulus but reduced by the Seoul Square image stimulus.

In the case of relative beta waves, an EEG average was not changed by the Seoul Square image stimulus but reduced by the Galleria Dept. image stimulus.

In the case of relative gamma waves, an EEG average was reduced by both the Galleria Dept. image stimulus and the Seoul Square image stimulus, so that the relative gamma waves were not activated. It is analyzed that an EEG average resulting from the Galleria Dept. image stimulus was more reduced than an EEG average resulting from the Seoul Square image stimulus.

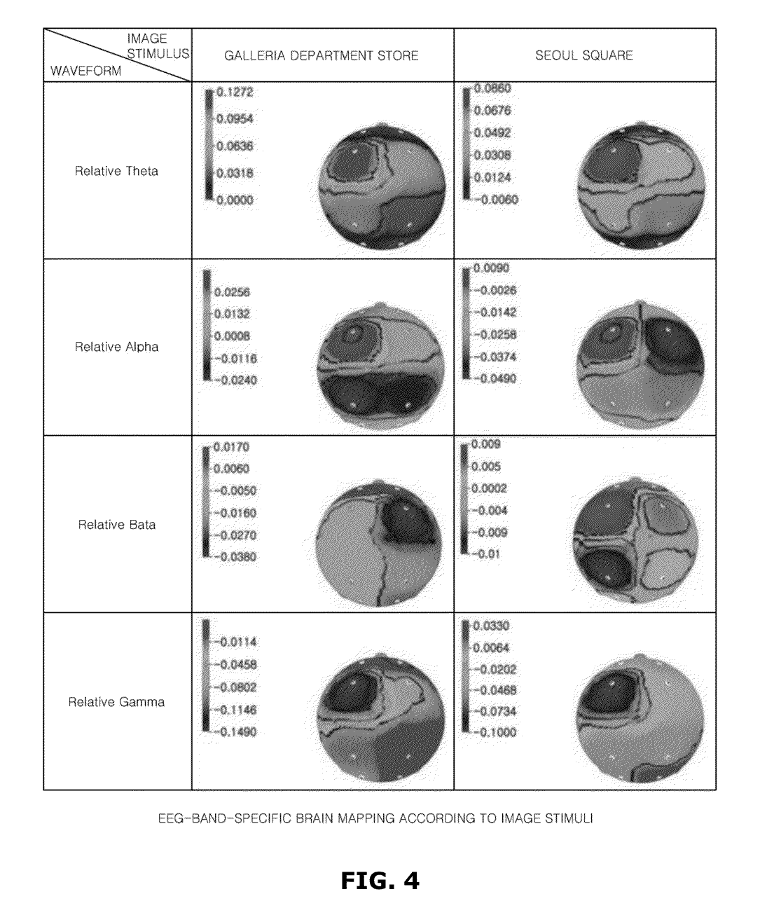

As shown in FIG. 4, the EEG band activity analysis unit 134 generates EEG-band-specific brain mapping image data according to experimental image stimuli based on the power amount of a specific frequency band received from the control unit 131.

For example, it is possible to generate a brain map including EEG potentials using BrainMap-3D.

Brain mapping image data visually shows which region of a subject's brain has been activated by an image stimulus.

In the analysis results of brain mapping images (FIG. 4), bands in which brain regions activated by the experimental image stimuli are similar to each other are distinguished from bands in which brain regions are significantly different from each other.

In the theta wave and gamma wave bands, there were differences in value, but activated regions were shown as similar patterns. First, in the theta wave band, the left frontal lobe was activated the most, and the prefrontal lobes and the occipital lobes had low EEG averages.

In the gamma wave band, the left frontal lobe had the lowest EEG average, and the right occipital lobe was activated the most.

Alpha waves and beta waves show differences in activated regions. First, in the alpha wave band, activities of the right frontal lobe were similar to each other. However, while the Galleria Dept. image stimulus reduced the EEG averages of the parietal lobes, the Seoul Square image stimulus remarkably reduced the EEG average of the right frontal lobe.

In the beta wave band, the Galleria Dept. image stimulus activated the prefrontal lobes and reduced the EEG average of the right frontal lobe, the Seoul Square image stimulus activated the prefrontal lobes and the left frontal lobe and reduced the EEG average of the left parietal lobe.

As shown in FIG. 4, brain mapping image data shows high EEG averages in colors close to red, which indicate activated brain regions, and shows low EEG averages in colors close to violet.

Through the overall analysis of changes in band-specific EEG averages (Table 2) and activities of brain regions, it was confirmed that an image stimulus of a media facade may stimulate a subject's emotion and feeling.

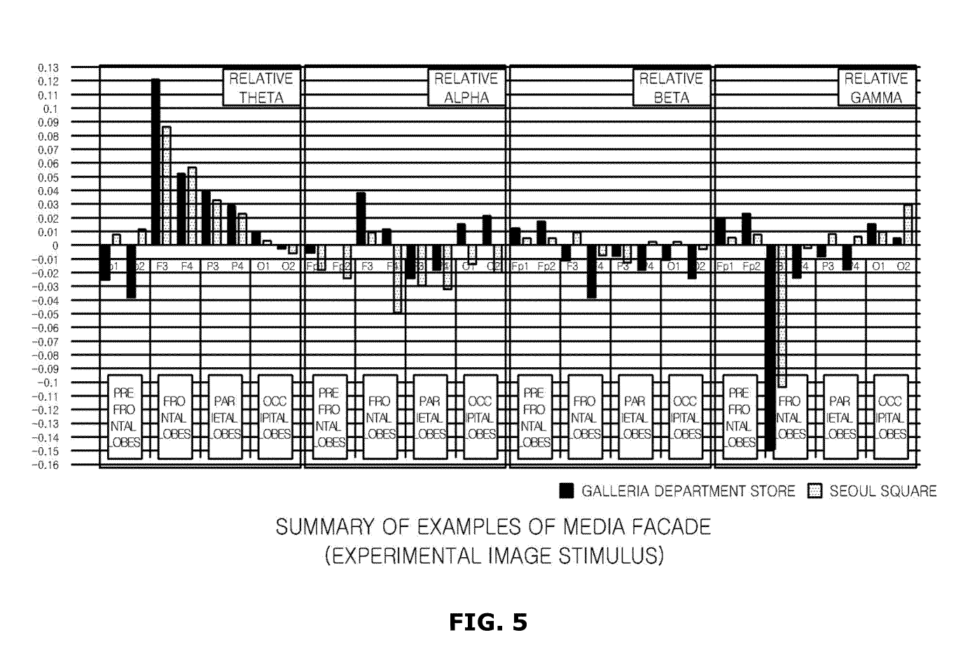

FIG. 5 is a histogram showing changes in activities of EEG bands dependent on brain regions according to an exemplary embodiment of the present disclosure.

The EEG band activity analysis unit 134 calculates EEG averages of the EEG electrode channels dependent on respective brain regions in the respective EEG bands according to the types of experimental image stimuli, comparatively analyzes brain-region-specific activity ratios of the EEG bands (Table 3 and FIG. 5).

The EEG band activity analysis unit 134 stores the analysis results in the memory unit 133 or outputs the analysis results to the display unit 135.

The EEG band activity analysis unit 134 determines activities according to the brain regions by calculating the EEG averages of the EEG electrode channels in the respective EEG bands according to the first image stimulus or the second image stimulus, and analyzes image stimulus results of the first image stimulus and the second image stimulus exerted on the subjects by analyzing changes in and differences between the EEG averages of the EEG electrode channels resulting from the first image stimulus and the second image stimulus.

More specifically, changes in region-specific activities of the EEG bands are analyzed, and statistical significance levels of EEG averages between examples are tested through a paired sample T-test, so that differences are examined in Table 3 below.

TABLE 3

| | | | Stimulus-specific | | | | |
|---|---|---|---|---|---|---|---|
| | | | EEG averages (SD) | | | | |
| | | | (Background stimu- | | | | |
| | | | lus − image stimulus) | | Difference | | |
| | | | Galleria de- | Seoul | in average | t | p |
| Waveform | EEG channel | | partment store | Square | (D) | value | value |
| Relative | Prefrontal | Fp1 | −0.025 | 0.007 | −0.032 | −0.906 | 0.369 |
| theta | lobe | | (0.232) | (0.240) | | | |
| | | Fp2 | −0.038 | 0.011 | −0.049 | −1.461 | 0.150 |
| | | | (0.252) | (0.251) | | | |
| | Frontal | F3 | 0.121 | 0.086 | 0.035 | 1.371 | 0.176 |
| | lobe | | (0.221) | (0.200) | | | |
| | | F4 | 0.052 | 0.057 | −0.005 | −0.175 | 0.862 |
| | | | (0.190) | (0.176) | | | |
| | Parietal | P3 | 0.040 | 0.033 | 0.007 | 0.356 | 0.724 |
| | lobe | | (0.131) | (0.118) | | | |
| | | P4 | 0.028 | 0.023 | 0.005 | 0.195 | 0.846 |
| | | | (0.150) | (0.146) | | | |
| | Occipital | O1 | 0.010 | 0.003 | 0.007 | 0.364 | 0.717 |
| | lobe | | (0.119) | (0.105) | | | |
| | | O2 | −0.003 | −0.006 | 0.003 | 0.153 | 0.879 |
| | | | (0.149) | (0.103) | | | |
| Relative | Prefrontal | Fp1 | −0.006 | −0.018 | 0.012 | 0.784 | 0.436 |
| alpha | lobe | | (0.070) | (0.104) | | | |
| | | Fp2 | 0.000 | −0.024 | 0.024 | 1.395 | 0.169 |
| | | | (0.080) | (0.105) | | | |
| | Frontal | F3 | 0.038 | 0.009 | 0.029 | 1.408 | 0.165 |
| | lobe | | (0.172) | (0.142) | | | |
| | | F4 | 0.011 | −0.049 | 0.06 | 2.558 | 0.013* |
| | | | (0.163) | (0.146) | | | |
| | Parietal | P3 | −0.024 | −0.030 | 0.006 | 0.244 | 0.808 |
| | lobe | | (0.190) | (0.157) | | | |
| | | P4 | −0.018 | −0.032 | 0.014 | 0.604 | 0.548 |
| | | | (0.184) | (0.164) | | | |
| | Occipital | O1 | 0.015 | −0.014 | 0.029 | 0.983 | 0.330 |
| | lobe | | (0.184) | (0.146) | | | |
| | | O2 | 0.021 | −0.020 | 0.041 | 1.418 | 0.162 |
| | | | (0.191) | (0.194) | | | |
| Relative | Prefrontal | Fp1 | 0.012 | 0.005 | 0.007 | 0.398 | 0.692 |
| beta | lobe | | (0.110) | (0.114) | | | |
| | | Fp2 | 0.017 | 0.005 | 0.012 | 0.729 | 0.469 |
| | | | (0.140) | (0.129) | | | |
| | Frontal | F3 | −0.011 | 0.009 | −0.02 | −1.659 | 0.103 |
| | lobe | | (0.111) | (0.110) | | | |
| | | F4 | −0.038 | −0.007 | −0.031 | −2.373 | 0.021* |
| | | | (0.098) | (0.093) | | | |
| | Parietal | P3 | −0.008 | −0.013 | 0.005 | 0.402 | 0.689 |
| | lobe | | (0.086) | (0.085) | | | |
| | | P4 | −0.018 | 0.002 | −0.02 | −1.718 | 0.091 |
| | | | (0.085) | (0.090) | | | |
| | Occipital | O1 | −0.011 | 0.002 | −0.013 | −0.688 | 0.494 |
| | lobe | | (0.103) | (0.095) | | | |
| | | O2 | −0.024 | −0.003 | −0.021 | −1.291 | 0.202 |
| | | | (0.106) | (0.093) | | | |
| Relative | Prefrontal | Fp1 | 0.020 | 0.005 | 0.015 | 0.943 | 0.350 |
| gamma | lobe | | (0.113) | (0.093) | | | |
| | | Fp2 | 0.023 | 0.007 | 0.016 | 1.193 | 0.238 |
| | | | (0.101) | (0.099) | | | |
| | Frontal | F3 | −0.149 | −0.104 | −0.045 | −2.257 | 0.028* |
| | lobe | | (0.269) | (0.247) | | | |
| | | F4 | −0.024 | −0.002 | −0.022 | −1.157 | 0.252 |
| | | | (0.149) | (0.141) | | | |
| | Parietal | P3 | −0.008 | 0.008 | −0.016 | −1.191 | 0.239 |
| | lobe | | (0.114) | (0.111) | | | |
| | | P4 | −0.018 | 0.006 | 0.002 | −0.741 | 0.462 |
| | | | (0.184) | (0.112) | | | |
| | Occipital | O1 | 0.015 | 0.009 | −0.025 | 0.204 | 0.839 |
| | lobe | | (0.184) | (0.083) | | | |
| | | O2 | 0.005 | 0.029 | −0.024 | −1.075 | 0.287 |
| | | | (0.178) | (0.156) | | | |

*p < 0.05

First, examination results of changes in band-specific EEG averages indicate that EEG averages resulting from the Galleria Dept. image stimulus and the Seoul Square image stimulus show a variety of differences according to bands and regions. Electrodes capable of measuring EEGs are classified according to brain regions. EEG averages of eight electrode channels (prefrontal lobe(left): Fp1, prefrontal lobe(right): Fp2, frontal lobe(left): F3, frontal lobe(right): F4, parietal lobe(left): P3, parietal lobe(right): P4, occipital lobe(left): O1, and occipital lobe(right): O2) among the total of 32 electrode channels were used in the analysis.

Referring to respective averages indicating activities of the EEG electrode channels measured in these regions according to the regions, EEG averages (i.e., activities) of the prefrontal lobes Fp1 and Fp2 and the occipital lobe O2 resulting from the Galleria Dept. image stimulus were reduced in the theta wave bands compared to background EEG averages, and EEG averages of the other channel regions were increased. In particular, EEGs of the frontal lobes were activated the most.

All EEG averages of channels resulting from the Seoul Square other than that of the occipital lobe O2 were increased.

A common characteristic of changes in EEG averages resulting from the Galleria Dept. image stimulus and the Seoul Square image stimulus is in that there are differences in EEG activities of the frontal lobes F3 and F4, the parietal lobes P3 and P4, and the occipital lobe O1. In particular, there are notable differences in the prefrontal lobes Fp1 and Fp2 that are very activated while processing new information but are remarkably reduced in activity after gaining familiarity therewith.

Referring to differences according to waveforms, the number of channels of relative alpha waves whose EEG averages were reduced was larger than the number of channels of relative alpha waves whose EEG averages were increased. In a channel whose EEG average resulting from the Seoul Square image stimulus is lower than an EEG average resulting from a background image stimulus, a reduction in the EEG average resulting from the Seoul Square image stimulus was larger than the EEG average resulting from the Galleria Dept. image stimulus.

In the case of the Galleria Dept. image stimulus, relative alpha-wave averages of the frontal lobes F3 and F4 and the occipital lobes O1 and O2 were increased, and in the case of the Seoul Square image stimulus, only a relative alpha-wave average of the frontal lobe F3 was increased. In the two cases, relative alpha-wave averages of the parietal lobes P3 and P4 were reduced in common, and contradictory activities were shown in the occipital lobes O1 and O2.

Compared to the other EEG bands, the degrees of changes in the relative beta wave band were the slightest. Channel regions whose changes in EEG averages resulting from the two stimuli were opposite to each other were the frontal lobe F3, the parietal lobe P4, and the occipital lobe O1.

In addition to these, EEG averages of the prefrontal lobes Fp1 and Fp2 were increased together, and EEG averages of the frontal lobe F4, the parietal lobe P3, and the occipital lobe O2 were reduced together.

It is analyzed that EEG averages resulting from the Seoul Square image stimulus exhibited little change in the occipital lobes.

In the case of the relative gamma wave band, EEG averages of the prefrontal lobes Fp1 and Fp2 and the occipital lobes O1 and O2 resulting from the two stimuli were increased together, and EEG averages of the frontal lobes F3 and F4 were reduced together. In particular, an EEG average of the frontal lobe F3 was reduced by the largest amount compared to the EEG average of the entire bands, and showed a notable difference in the opposite direction of an EEG value of the frontal lobe F3 in the theta wave band.

In the parietal lobes P3 and P4, EEG averages resulting from the two stimuli showed differences. While EEG averages resulting from the Galleria Dept. image stimulus were reduced, EEG averages resulting from the Seoul Square were increased, so that the parietal lobes P3 and P4 were activated.

As shown in the analysis results, EEG averages of electrode channels showed differences in each EEG band. However, channels showing statistically significant differences were the frontal lobe F4 in the alpha wave band (t=2.558, p<0.5), the frontal lobe F4 in the beta wave band (t=−2.373, p<0.5), and the frontal lobe F3 in the gamma wave band (t=−2.257, p<0.5), and it is analyzed that there were differences in the frontal lobes in common.

On the other hand, there was not any channel showing a statistically significant difference in the theta wave band.

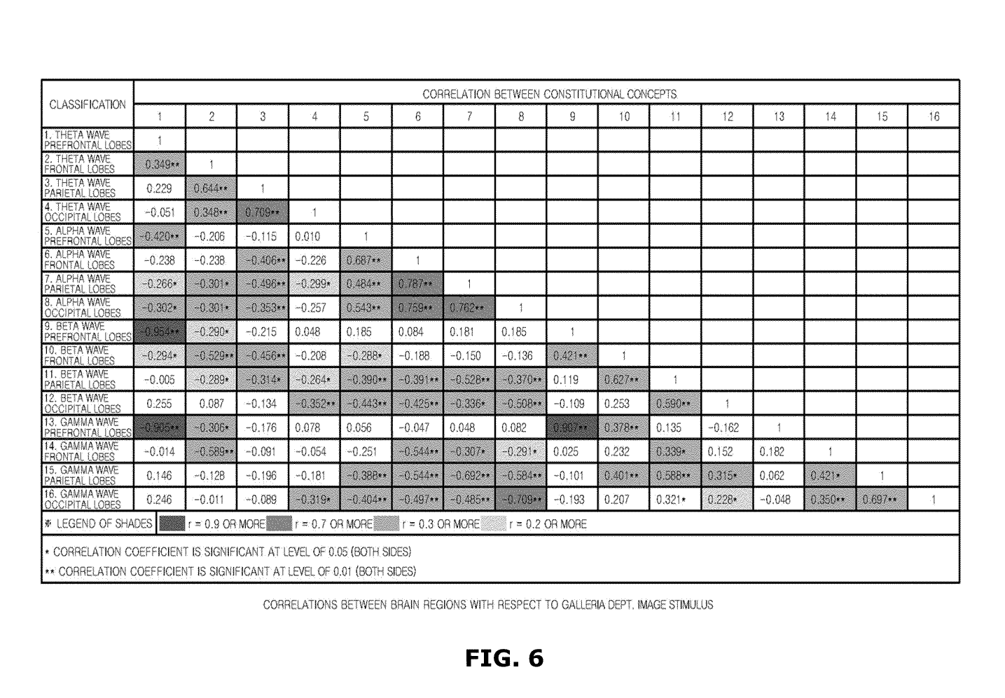

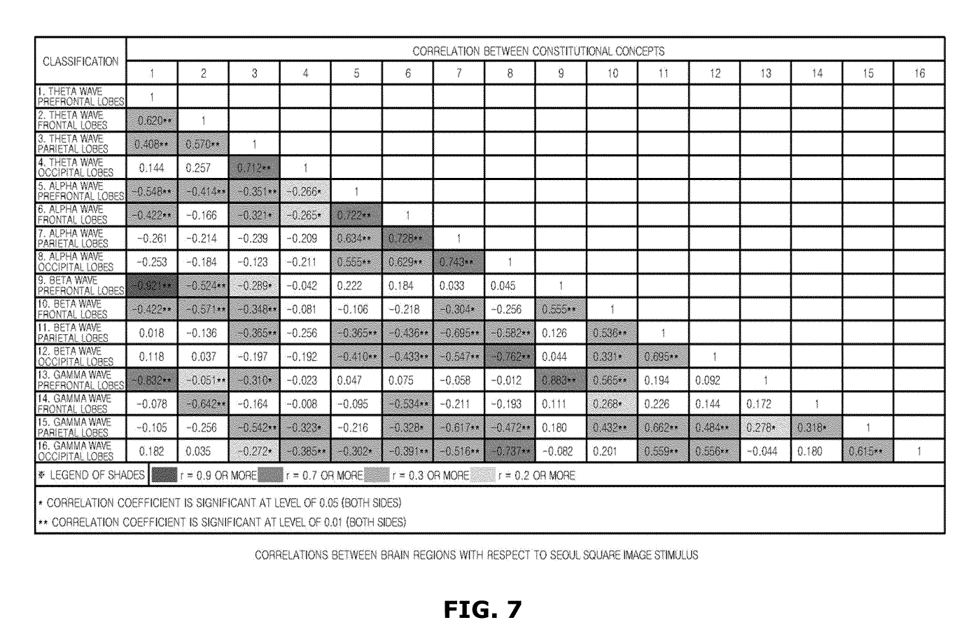

Next, referring to FIGS. 6 and 7, correlations between brain regions with respect to an image stimulus will be analyzed according to the EEG bands.

FIG. 6 is a diagram in which correlations between brain regions with respect to an image stimulus of a Galleria department store are analyzed according to an exemplary embodiment of the present disclosure, and FIG. 7 is a diagram in which correlations between brain regions with respect to an image stimulus of Seoul Square are analyzed according to an exemplary embodiment of the present disclosure.

The EEG band activity analysis unit 134 analyzes correlations between calculated EEG averages of the EEG electrode channels.

The EEG band activity analysis unit 134 stores the correlation analysis results in the memory unit 133 or outputs the correlation analysis results to the display unit 135.

(1) Galleria Dept. Image Stimulus

As shown in FIG. 6, to process information on the image stimulus of the media facade of the Galleria department store, correlations between EEG averages measured from four brain regions, that is, the prefrontal lobes, the frontal lobes, the parietal lobes, and the occipital lobes, were analyzed according to the EEG bands. As a result, a plurality of regions showing statistically significant correlations with r (correlation coefficient) of 0.5 or more were found.

Referring to EEG-band-specific brain regions showing very high correlations with r of 0.9 or more, there were a total of two pairs of EEG-band-specific brain regions showing a negative correlation, that is, a relationship in which one region is deactivated when the other region is activated. The two pairs were a pair of a beta-wave prefrontal lobe and a theta-wave prefrontal lobe (r=−0.954, p<0.01) and a pair of a gamma-wave prefrontal lobe and a theta-wave prefrontal lobe (r=−0.905, p<0.01).

On the other hand, a pair of a gamma-wave prefrontal lobe and a beta-wave prefrontal lobe (r=0.907, p<0.01) showed a statistically very highly significant positive correlation and was simultaneously activated by the stimulus.

In the analysis results, it is to be noted that statistically very highly significant correlations (r is 0.9 or more) resulting from the Galleria Dept. image stimulus were shown between only the prefrontal lobes in different EEG bands.

Also, an EEG band including the largest number of pairs of brain regions showing a high accordance (correlation) with r of 0.7 or more between left and right cerebral hemispheres was the alpha wave band. Among a total of five correlation pairs, three correlation pairs (a pair of an alpha-wave parietal lobe and an alpha-wave frontal lobe (r=0.787, p<0.01), a pair of an alpha-wave occipital lobe and the alpha-wave frontal lobe (r=0.759, p<0.01), and a pair of the alpha-wave occipital lobe and the alpha-wave parietal lobe (r=0.762, p<0.01)) were shown in the alpha wave band.

Further, analysis results of EEG-band-specific relationships between the occipital lobes indicate that measured regions showing a high correlation (r of 0.7 or more) with respect to the Galleria Dept. image stimulus were a pair of the alpha-wave occipital lobe and a gamma-wave occipital lobe (r=−0.709, p<0.01).

Finally, among a total of 120 measured regions, 45 regions showed statistically significant correlations (significance level p<0.05) with respect to the Galleria Dept. image stimulus (FIG. 6).

(2) Seoul Square Image Stimulus

As shown in FIG. 7, regions showing a statistically very highly significant correlation with r of 0.9 or more were only one pair of a beta-wave prefrontal lobe and a theta-wave prefrontal lobe (r=−0.921, p<0.01). The regions showed the negative correlation, and thus it is analyzed that activation was caused by the stimulus contradictorily in the both regions.

Measured regions showing high correlations with r of 0.8 or more were a pair of a gamma-wave prefrontal lobe and the theta-wave prefrontal lobe (r=−0.832, p<0.01) and a pair of the gamma-wave prefrontal lobe and a relative beta-wave prefrontal lobe (r=0.883, p<0.01).

Like the results of the Galleria Dept. image stimulus, an EEG band including the largest number of pairs of brain regions showing a high accordance (correlation) with r of 0.7 or more between left and right cerebral hemispheres was the alpha wave band (a pair of an alpha-wave frontal lobe and an alpha-wave prefrontal lobe (r=0.722, p<0.01), a pair of an alpha-wave parietal lobe and the alpha-wave frontal lobe (r=0.728, p<0.01), and a pair of an alpha-wave occipital lobe and the alpha-wave parietal lobe (r=0.743, p<0.01)).

Analysis results of relationships between EEG-band-specific occipital lobes indicate that measured regions showing a high correlation (r of 0.7 or more) were a pair of a beta-wave occipital lobe and an alpha-wave occipital lobe (r=−0.762, p<0.01) and a pair of a gamma-wave occipital lobe and the alpha-wave occipital lobe (r=−0.737, p<0.01).

The Seoul Square image stimulus resulted in a larger number of measured regions showing statistically significant correlations (significance level p<0.05) than the Galleria Dept. image stimulus.

It is confirmed that the subjects required more brain regions to be simultaneously connected when information on the Seoul Square image stimulus was processed.

Finally, among a total of 120 measured regions, 50 regions showed statistically significant correlations (significance level p<0.05) with respect to the Seoul Square image stimulus (FIG. 7).

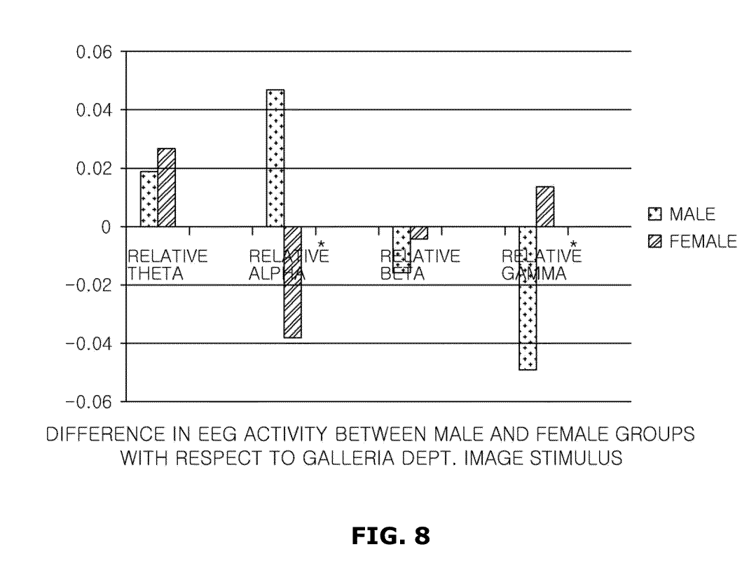

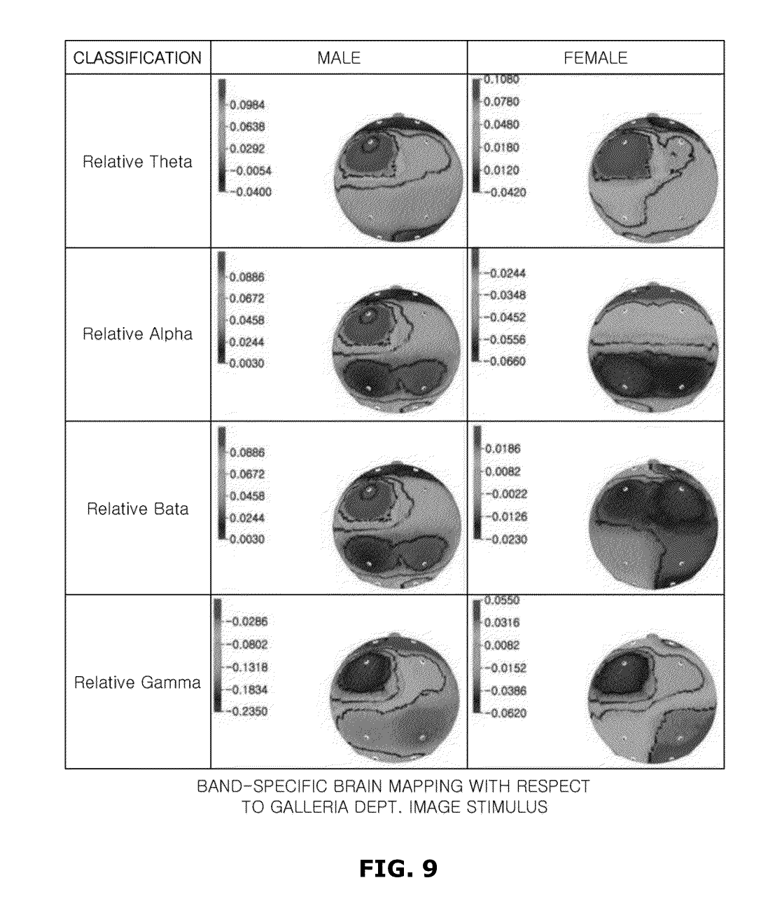

Next, EEG activities of male and female groups caused by the Galleria Dept. image stimulus will be analyzed with reference to FIGS. 8 and 9.

FIG. 8 is a diagram showing differences in EEG activities caused by the Galleria Dept. image stimulus between male and female groups according to an exemplary embodiment of the present disclosure, and FIG. 9 is a diagram showing EEG-band-specific brain mapping image data resulting from the Galleria Dept. image stimulus according to an exemplary embodiment of the present disclosure.

Results of analyzing differences in EEG activity between both sexes through an independent sample T-test indicate that there were differences in EEG averages resulting from the Galleria Dept. and Seoul Square image stimuli.

EEG averages resulting from the Galleria Dept. image stimulus showed differences in all the EEG bands. While the relative theta wave band was activated in both the male and female groups, EEG averages were reduced in the beta wave band.

While the alpha wave band was activated in the male group because an EEG average was increased, an EEG average of the female group was reduced in the alpha wave band. While the gamma wave band was activated in the female group, an EEG average of the male group was reduced in the gamma wave band.

Meanwhile, it is analyzed that EEG bands showing differences in EEG average between the male and female groups at statistically significant levels (p<0.05) were the alpha wave band (male(M=0.047), female(M=−0.038)) and the gamma wave band (male(M=−0.049), female(M=0.014)).

EEG averages of both the male and female groups resulting from the Seoul Square image stimulus were increased in the relative theta wave band, and the relative theta wave band was activated. While the relative alpha wave band was activated in the male group, an EEG average of the female group was reduced.

While EEG averages of the male group were reduced in the relative beta wave and relative gamma wave bands, those of the female group were increased, and the relative beta wave and relative gamma wave bands were slightly activated.

However, EEG averages resulting from the Seoul Square image stimulus did not show a statistically significant difference.

Meanwhile, referring to brain regions activated by the Galleria Dept. image stimulus through brain mapping, the male and female groups showed similar patterns in the theta wave and gamma wave bands but showed clear differences between regions activated in the alpha and beta wave bands.

First, in the alpha wave band, while the male group showed an activated left frontal lobe and remarkably reduced EEG averages of the prefrontal lobes, the female group showed remarkably activated prefrontal lobes and remarkably reduced EEG averages of the parietal lobes.

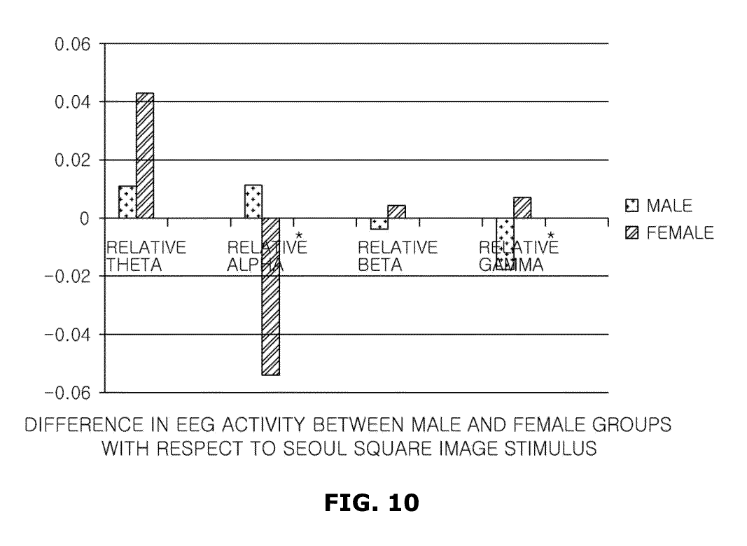

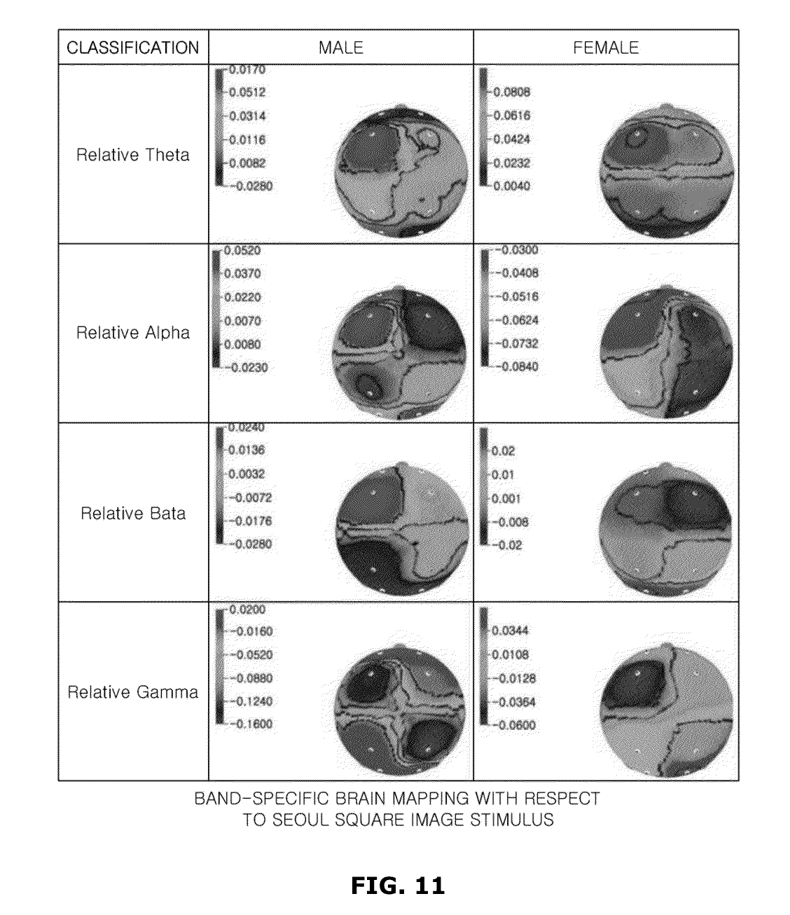

Next, EEG activities of male and female groups caused by the Seoul Square image stimulus will be analyzed with reference to FIGS. 10 and 11.

FIG. 10 is a diagram showing differences in EEG activities caused by the Seoul Square image stimulus between male and female groups according to an exemplary embodiment of the present disclosure, and FIG. 11 is a diagram showing EEG-band-specific brain mapping image data resulting from the Seoul Square image stimulus according to an exemplary embodiment of the present disclosure.

The male and female groups showed different activities of brain regions overall with respect to the Seoul Square image stimulus. First, in the theta wave band, while the male group showed the activated left frontal lobe and remarkably reduced EEG averages of the prefrontal lobes, the female group showed the activated left and right frontal lobes and reduced EEG averages of the occipital lobes.

In the alpha wave band, while the male group showed the activated left frontal lobe and a remarkably reduced EEG average of the right frontal lobe, the female group showed the remarkably activated prefrontal lobes and left frontal lobe and a remarkably reduced EEG average of the right occipital lobe.

In the beta wave band, while the male group showed the activated left frontal lobe, the female group showed the activated occipital lobes.

Finally, in the gamma wave band, while the male group showed the remarkably activated prefrontal lobes, right frontal lobe, left parietal lob, and occipital lobes, the female group showed the activated right occipital lobe and a remarkably reduced EEG average of the left frontal lobe.

Comparative analysis of brain-region-specific activities of the male and female groups is as shown in Table 4 below.

TABLE 4
Analysis of differences in brain-region-specific
activities between male and female groups (N = 56)

| | | | Stimulus-specific | | | | |
|---|---|---|---|---|---|---|---|
| | | | EEG averages (SD) | | | | |
| | | | (Background stimu- | | Difference | | |
| | | | lus − image stimulus) | | in average | t | p |
| Waveform | | | Male | Female | (D) | value | value |
| Galleria | Relative | F3 | 0.110 | −0.033 | 0.143 | 3.378 | 0.001* |
| | alpha | | (0.100) | (0.099) | | | |
| | | F4 | 0.054 | −0.032 | 0.086 | 2.034 | 0.047* |
| | | | (0.187) | (0.123) | | | |
| | | O1 | 0.067 | −0.038 | 0.105 | 2.202 | 0.032* |
| | | | (0.201) | (0.151) | | | |
| | | O2 | 0.086 | −0.044 | 0.130 | 2.698 | 0.009* |
| | | | (0.183) | (0.187) | | | |
| | Relative | F3 | −0.235 | −0.062 | −0.173 | −2.518 | 0.015* |
| | gamma | | (0.315) | (0.180) | | | |
| | | O1 | −0.051 | 0.019 | −0.07 | −2.213 | 0.031* |
| | | | (0.124) | (0.113) | | | |
| | | O2 | −0.045 | 0.055 | −0.1 | −2.156 | 0.036* |
| | | | (0.164) | (0.180) | | | |
| Seoul | Relative | F3 | 0.052 | −0.033 | 0.085 | 2.371 | 0.021* |
| Square | alpha | | (0.141) | (0.131) | | | |
| | | O1 | 0.031 | −0.059 | 0.09 | 2.411 | 0.019* |
| | | | (0.142) | (0.138) | | | |
| | | O2 | 0.043 | −0.084 | 0.127 | 2.575 | 0.013* |
| | | | (0.205) | (0.162) | | | |
| | Relative | O1 | −0.026 | 0.029 | −0.055 | −2.252 | 0.028* |
| | beta | | (0.098) | (0.084) | | | |
| | | O2 | −0.028 | 0.022 | −0.05 | −2.045 | 0.046* |
| | | | (0.089) | (0.092) | | | |

*Only band-specific averages of regions showing statistically significant differences are shown.
*p < 0.05

Average band-to-band powers of the male and female groups resulting from the Galleria Dept. image stimulus showed statistically significant differences in the alpha wave and gamma wave bands.

Specifically, in the alpha wave band, averages of the male and female groups showed differences in the F3 (t=3.378, p<0.05) and F4 (t=2.034, p<0.05) channels of the frontal lobes and the O1 (t=2.202, p<0.05) and O2 (t=2.698, p<0.05) channels of the occipital lobes.

Referring to the averages of the channels showing statistically significant differences in the alpha wave band, the channels of the male group were activated in common, and EEG averages of the female group were reduced compared to before provision of the image stimulus.

In the gamma wave band, averages of the male and female groups showed differences in the F3 (t=−2.518, p<0.05) channel of the frontal lobe and the O1 (t=−2.213, p<0.05) and O2 (t=−2.156, p<0.05) channels of the occipital lobes.

Referring to averages of the channels showing statistically significant differences in the gamma wave band, the EEG averages of the male group were reduced in common compared to before provision of the image stimulus, and the O1 and O2 channels of the occipital lobes of the female group excluding the F3 channel were activated.

Average band-to-band powers of the male and female groups resulting from the Seoul Square image stimulus showed statistically significant differences in the alpha wave and beta wave bands.

Specifically, in the alpha wave band, averages of the male and female groups showed differences in the F3 (t=2.371, p<0.05) channel of the frontal lobe and the O1 (t=2.411, p<0.05) and O2 (t=2.575, p<0.05) channels of the occipital lobes.

Referring to averages of the channels showing statistically significant differences in the alpha wave band, the channels of the male group were activated in common, and EEG averages of the female group were reduced compared to before provision of the image stimulus.

These results show the same tendency as changes in EEG averages resulting from the Galleria Dept. image stimulus, and it is estimated that such changes in EEGs were made because the male and female groups were influenced not by expressive types or content of the image stimuli but by the stimuli which are usually difficult to see.

In the beta wave band, only the occipital lobes showed statistically significant differences. In the O1 (t=−2.252, p<0.05) and O2 (t=−2.045, p<0.05) channels, the male and female groups showed the differences.

Referring to averages of the channels showing the statistically significant differences in the beta wave band, while EEG averages of the male group were reduced in common compared to before provision of the image stimulus, EEG averages of the female group were increased, and the channels of the female group were activated.

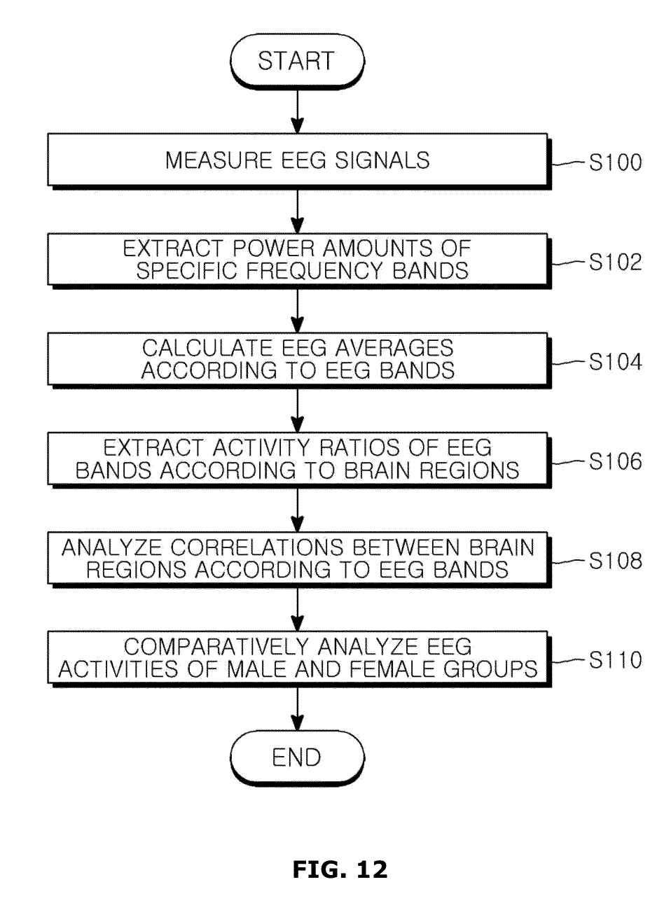

FIG. 12 is a flowchart illustrating a method of analyzing an EEG response to an image stimulus of a media facade according to an exemplary embodiment of the present disclosure.

The EEG sensing unit 110 measures EEG signals from the EEG electrode channels installed in respective brain regions of a subject who is viewing an experimental image stimulus (S100).

The FFT unit 132 converts the measured EEG signals from the time domain to the frequency domain.

The control unit 131 extracts wavelengths of a specific frequency band from the converted frequency-domain EEG signals, and extracts frequency-specific power amounts using frequency-specific power spectrums (S102).

The EEG band activity analysis unit 134 calculates EEG-band-specific EEG averages, which represent EEG-band-specific activity ratios based on the average of band-to-band powers, based on the extracted frequency-specific power amounts as shown in Table 2 and FIG. 4 (S104), and calculates EEG averages of the EEG electrode channels in the respective EEG bands according to the brain regions to comparatively analyze brain-region-specific activity ratios of the EEG bands as shown in Table 3 and FIG. 5 (S106).

As shown in FIGS. 6 and 7, the EEG band activity analysis unit 134 determines brain-region-specific correlations of EEG bands by analyzing correlations between EEG averages of the EEG electrode channels according to the EEG bands (S108).

As shown in FIGS. 8 to 11, the EEG band activity analysis unit 134 calculates EEG averages of male and female groups of subjects according to the EEG bands and comparatively analyzes EEG activities of the respective male and female groups (S110).

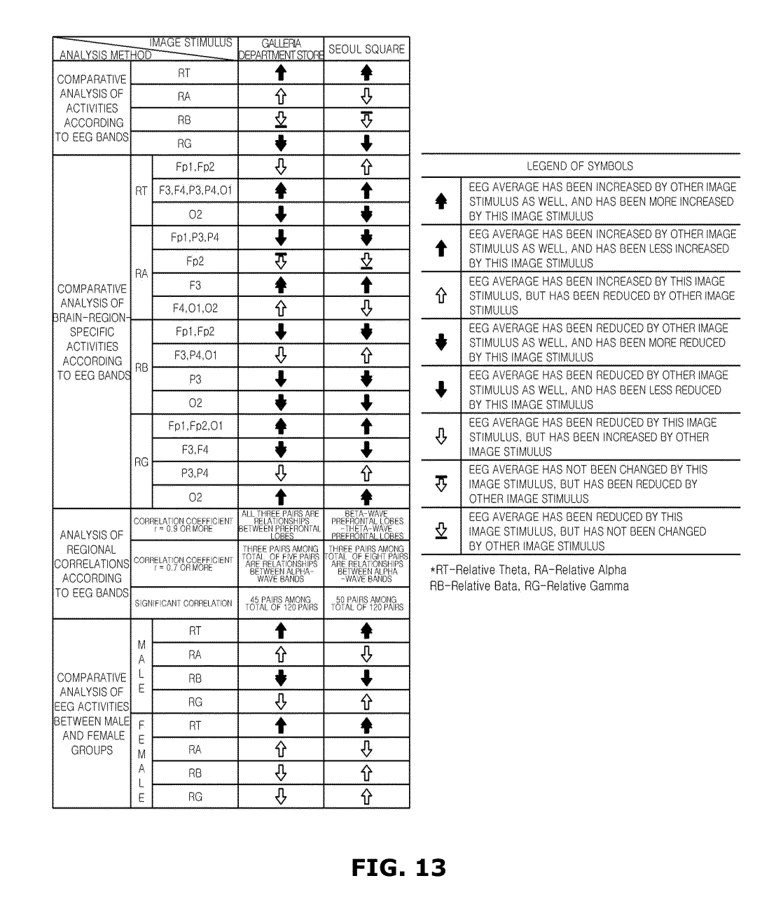

Next, comprehensively arranged EEG analysis results of subjects with respect to the image stimuli of the Galleria department store and Seoul Square will be described with reference to FIG. 13.

FIG. 13 is a table showing comprehensively arranged EEG analysis results of subjects with respect to the Galleria Dept. and Seoul Square image stimuli according to an exemplary embodiment of the present disclosure.

First, comparative analysis results of EEG-band-specific activities indicate that the theta wave band was notably activated in common compared to the other EEG bands.

In general, theta waves are known to mainly relate to emotions and feelings and to be involved in a calm physical state, emotions, and mental activities.

From these research results, it is possible to infer that an image stimulus of a media facade configured with a variety of colors and shapes stimulates creativity and emotions of a subject.

While the alpha wave band was activated by the Galleria Dept. image stimulus, EEG averages were reduced by the Seoul Square image stimulus.

Thus far, researchers who have researched changes in EEGs caused by visual stimuli have provided a research result indicating that an increase in visual attention results in a significant reduction in alpha waves.

From the research results, it is possible to infer that the Seoul Square image stimulus configured with image content attracted more attention than the Galleria Dept. image stimulus configured with simple changes in color and shape.

This may be described with the changes in beta waves. The Galleria Dept. image stimulus stimulated less beta waves than the Seoul Square image stimulus. When a task requiring concentration is assigned to a normal person, there occurs an EEG change described as desynchronization or alpha-wave suppression, and rapid brainwaves referred to as beta waves occurs.

In addition, both beta waves and gamma waves were reduced. From this fact, it is possible to see that neither of the two image stimuli had a significantly negative influence, such as stress or anxiety, on the subjects.

Gamma waves increase when a task requiring a high cognitive function is performed.

Since neither of the Galleria Dept. image stimulus and the Seoul Square image stimulus require a high cognitive capability or calculation and reasoning abilities, gamma waves are not activated. It is possible to determine that negative emotions, such as stress, were not aroused for this reason.

Comparative analysis results of activities of brain-region-specific electrode channels indicate that EEG changes of the prefrontal lobes showed contradictory results in the theta wave band. While an EEG resulting from the Seoul Square image stimulus was activated, an EEG resulting from the Galleria Dept. image stimulus was deactivated. The prefrontal lobes are very activated while processing new information but are remarkably reduced in activity after gaining familiarity therewith.

It is possible to determine that the Galleria Dept. image stimulus, which repeatedly shows the same pattern, and the Seoul Square image stimulus, which shows varying content over time, had an influence on adaptation to and familiarity with the stimuli.

In the alpha wave band, alpha waves of the parietal lobes were reduced in common, and the parietal lobes are in charge of emotional reactions. In other words, although it is not possible to accurately verify which aspect of an image stimulus of a media facade has an influence on subjects' emotions, it is possible to infer that the image stimulus may be a medium causing emotional reactions at least.

The occipital lobes are visual centers associated with a role in looking at an object, and EEGs resulting from the two image stimuli showed contradictory results.

While an EEG was activated by the Galleria Dept. image stimulus, an EEG was deactivated by the Seoul Square image stimulus. The Galleria Dept. image stimulus is an image in which a color arrangement is clearly shown and a clear shape is repeated, and thus it is possible to infer that the contradictory results were obtained because the Galleria Dept. image stimulated the subjects more than the Seoul Square image in which colors are not clearly distinguished.

In the beta and gamma wave bands, overall EEG changes were slight in common compared to the other bands, and the prefrontal lobes were more activated than the other regions.

Analysis results of regional correlations according to the EEG bands indicate that brain regions showing a very high correlation with r of 0.9 or more with respect to the Galleria Dept. image stimulus were three pairs and those with respect to the Seoul Square image stimulus were one pair. All the pairs correspond to relationships between the prefrontal lobes, and it is confirmed that a high accordance (correlations) between the prefrontal lobes resulted from both the two image stimuli in common.

Also, many high correlations with r of 0.7 or more resulted from the Galleria Dept. image stimulus and the Seoul Square image stimulus in the alpha wave band compared to the other bands.

More regions showing a statistically significant correlation (significance level p<0.05) resulted from the Seoul Square image stimulus (50 pairs) compared to the Galleria Dept. image stimulus (45 pairs).

It is confirmed that the subjects required more brain regions to be simultaneously connected when information on the Seoul Square image stimulus was processed.

Finally, comparative analysis results of EEG activities of the male and female groups indicate that there are differences in EEG changes caused by the two stimuli between the male and female groups.

First, in the case of the Galleria Dept. image stimulus, it is analyzed that a difference in EEG average between the male and female groups was shown at a statistically significant level (p<0.05) in the alpha wave and gamma wave bands.

In both the male and female groups, theta waves were increased, and theta waves of the female groups were more activated than those of the male groups.

This may be supported by research results indicating that females generally show higher activities in regions associated with emotional processing than males. In the alpha wave band, the male group and the female group showed the largest difference in EEG activity.

Assuming that alpha waves are ERPs related to visual attention, it is possible to infer that, while the female group whose alpha waves were reduced paid attention to repetitive pattern changes and contrasting color configurations of the Galleria department store, the male group paid less attention after recognizing repetitive patterns.

Beta waves of both the male and female groups were reduced. While relative gamma waves of the male group were remarkably reduced, those of the female group were activated.

Using the above-described configuration, an exemplary embodiment of the present disclosure provides an emotional indicator which may be evaluated according to image content.

An exemplary embodiment of the present disclosure provides a methodology for implementing modern architecture designs that pursue interaction with people through experience and emotional stimuli by measuring brain reactions to image stimuli of media facades.

An exemplary embodiment of the present disclosure measures emotions from visual stimuli of media facade images to derive and evaluate architectural elements causing an emotional reaction in the field of architecture.

An exemplary embodiment of the present disclosure provides an analysis method for analyzing emotional and physiological reactions to image designs.

It will be apparent to those skilled in the art that various modifications can be made to the above-described exemplary embodiments of the present disclosure without departing from the spirit or scope of the disclosure. Thus, it is intended that the present disclosure covers all such modifications provided they come within the scope of the appended claims and their equivalents.

What is claimed is:

1. A system for analyzing an electroencephalogram (EEG) reaction to an image stimulus of a media facade, the system comprising:
an EEG sensing unit configured to measure EEG signal strengths emitted from a plurality of EEG electrode channels installed in a plurality of regions of a brain of a viewer who views an experimental image stimuli;
a fast Fourier transform (FFT) unit configured to convert the EEG signal strengths from a time domain to a frequency domain;
a control unit configured to extract wavelengths in a predetermined frequency band from the frequency domain of the EEG signal strengths and to extract a power amount for each frequency using a frequency-specific power spectrum; and
an EEG band activity analysis unit configured to calculate a first EEG average in response to EEG activity ratios per an EEG band with respect to an average of a band-to-band power value based on the power amount for each frequency, to calculate a second EEG average of the EEG electrode channels in response to the EEG band according to the plurality of regions of the brain, and to comparatively analyze brain-region-specific activity ratios of the EEG band.

2. The system of claim 1, wherein the EEG band includes a plurality of EEG frequencies including a relative theta wave of 4 to 8 Hz, a relative alpha wave of 8 to 13 Hz, a relative beta wave of 13 to 30 Hz, and a relative gamma wave equal or greater than 30 Hz in ascending order of frequency, and
the EEG electrode channels includes a left prefrontal lobe, a right prefrontal lobe, a left frontal lobe, a right frontal lobe, a left parietal lobe, a right parietal lobe, a left occipital lobe, and a right occipital lobe.

3. The system of claim 2, wherein the experimental image stimuli are three-dimensional (3D) images projected onto an inner or outer wall of a building, and include a first image stimulus configured with a pattern change in color and shape and a second image stimulus configured to use a change in color and shape for delivering a story as a storytelling medium.

4. The system of claim 3, wherein the EEG band activity analysis unit is configured to determine brain-region-specific activities by calculating the second EEG average of the EEG electrode channels depending on the EEG band according to the first image stimulus or the second image stimulus, and to analyze image stimulus results of the first image stimulus or the second image stimulus exerted on the viewer by analyzing changes in and differences between the second EEG average of the EEG electrode channels resulting from the first image stimulus or the second image stimulus.

5. The system of claim 3, wherein the EEG band activity analysis unit is configured to analyze correlations between the second EEG average of the EEG electrode channels and to determine that the prefrontal lobes are brain regions having a correlation coefficient of 0.9 or more between EEG electrode channels with respect to the first image stimulus and the second image stimulus.

6. The system of claim 3, wherein the EEG band activity analysis unit is configured to analyze the second EEG average of the EEG electrode channels and determine that a correlation between left and right cerebral hemispheres is shown in a relative alpha wave band rather than relative theta, beta, and gamma wave bands among brain regions showing a correlation coefficient of 0.7 or more between the EEG electrode channels with respect to the first image stimulus and the second image stimulus.

7. The system of claim 1, wherein at least one of the first EEG average and the second EEG average is calculated by subtracting image stimulus EEG averages of the experimental image stimuli from a background EEG average indicating a base EEG average resulting from a white image stimulus.

8. A method of analyzing an electroencephalogram (EEG) reaction to an image stimulus of a media facade, the method comprising:
measuring EEG signal strengths from a plurality of EEG electrode channels installed in a plurality of regions of a brain of a viewer who views an experimental image stimuli;
converting the EEG signal strengths from a time domain to a frequency domain using a fast Fourier transform (FFT) method;
extracting wavelengths in a predetermined frequency band from the frequency-domain of the EEG signal strengths, and extracting a power amount for each frequency using a frequency-specific power spectrums;
calculating a first EEG average in response to EEG activity ratios per an EEG band with respect to an average of a band-to-band power value based on the power amount for each frequency;
calculating a second EEG average of the EEG electrode channels in response to the EEG band according to the plurality of regions of the brain; and
comparatively analyzing brain-region-specific activity ratios of the EEG band.

9. The method of claim 8, wherein the EEG band includes a plurality of EEG frequencies including a relative theta wave of 4 to 8 Hz, a relative alpha wave of 8 to 13 Hz, a relative beta wave of 13 to 30 Hz, and a relative gamma wave equal or greater than 30 Hz in ascending order of frequency, and
the EEG electrode channels includes a left prefrontal lobe, a right prefrontal lobe, a left frontal lobe, a right frontal lobe, a left parietal lobe, a right parietal lobe, a left occipital lobe, and a right occipital lobe.

10. The method of claim 9, wherein the experimental image stimuli are three-dimensional (3D) images projected onto an inner or outer wall of a building, and include a first image stimulus configured with a pattern change in color and shape and a second image stimulus configured to use a change in color and shape for delivering a story as a storytelling medium,
brain-region-specific activities are determined by calculating the second EEG average of the EEG electrode channels depending on the EEG band according to the first image stimulus or the second image stimulus, and
image stimulus results of the first image stimulus or the second image stimulus exerted on the viewer by analyzing changes in and differences between the second EEG average of the EEG electrode channels resulting from the first image stimulus or the second image stimulus.

11. The method of claim 10, wherein the calculating of the first EEG average comprises generating the first image stimulus and the second image stimulus resulted from a highest EEG average of the left frontal lobe among the brain regions in a relative theta wave band, and resulted from a lowest EEG average of the left frontal lobe and a highest EEG average of the right occipital lobe among the brain regions in a relative gamma wave band, and
brain mapping image data visually showing which region of the brain has been activated is generated.

12. The method of claim 10, wherein the comparative analyzing of the brain-region-specific activity ratios comprises:
analyzing correlations between the second EEG average of the EEG electrode channels; and
finding that the prefrontal lobes are brain regions having a correlation coefficient of 0.9 or more between EEG electrode channels with respect to the first image stimulus and the second image stimulus.
